(19) United States
(12) Patent Application Publication
(10) Pub. No.: US 2024/0304329 A1
(43) Pub. Date: Sep. 12, 2024

(54) DYNAMIC PROMPT TUNING OF MACHINE LEARNING MODEL INPUTS

(71) Applicant: NEC Laboratories America, Inc., Princeton, NJ (US)

(72) Inventors: Wei Cheng, Princeton Junction, NJ (US); Haifeng Chen, West Windsor, NJ (US); Xujiang Zhao, Hillsborough, NJ (US); Xianjun Yang, Santa Barbara, CA (US)

(21) Appl. No.: 18/591,838

(22) Filed: Feb. 29, 2024

Related U.S. Application Data
(60) Provisional application No. 63/487,642, filed on Mar. 1, 2023.

(51) Int. Cl. G16H 50/20; G06F 40/40; G16H 20/00

(52) U.S. Cl. CPC G16H 50/20; G06F 40/40; G16H 20/00

(57) ABSTRACT

Methods and systems for prompt tuning include training a tuning function to set prompt position, prompt length, or prompt pool based on a language processing task. The tuning function is applied to an input query to generate a combined input, with prompt text having the prompt length, being selected according to the prompt pool, and being added to the input query at the prompt position. The combined input is applied to a language model.

This application claims priority to U.S. Patent Application No. 63/487,642, filed on Mar. 1, 2023, incorporated herein by reference in its entirety.

### BACKGROUND

### Technical Field

The present invention relates to language models and, more particularly, to tuning prompts for language models.

### Description of the Related Art

Large language models are versatile tools that can be used to handle a diverse set of natural language processing tasks, including natural language understanding and natural language generation. Additionally, a language model can be pretrained on a large set of unsupervised data, followed by supervised training on a smaller dataset for fine-tuning to a particular task. However, fine-tuning the entire pretrained language model can be inefficient, as such models may have many billions or trillions of parameters.

### SUMMARY

A method for prompt tuning includes training a tuning function to set prompt position, prompt length, or prompt pool based on a language processing task. The tuning function is applied to an input query to generate a combined input, with prompt text having the prompt length, being selected according to the prompt pool, and being added to the input query at the prompt position. The combined input is applied to a language model.

A system for prompt tuning includes a hardware processor and a memory that stores a computer program. When executed by the hardware processor, the computer program causes the hardware processor to train a tuning function to set prompt position, prompt length, or prompt pool based on a language processing task, to apply the tuning function to an input query to generate a combined input, with prompt text having the prompt length, being selected according to the prompt pool, and being added to the input query at the prompt position, and to apply the combined input to a language model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of dynamic prompt tuning for a language model, in accordance with an embodiment of the present invention;

FIG. 2 is a block/flow diagram of a method for training and using a prompt tuning function, in accordance with an embodiment of the present invention;

FIG. 3 is a block diagram of the use of a language model with prompt tuning in the context of a healthcare facility, in accordance with an embodiment of the present invention;

FIG. 4 is a block diagram of a computing system that can train and use a prompt tuning function, in accordance with an embodiment of the present invention;

FIG. 5 is a diagram of an exemplary neural network architecture that can be used to implement part of a prompt tuning function, in accordance with an embodiment of the present invention; and

FIG. 6 is a diagram of an exemplary neural network architecture that can be used to implement part of a prompt tuning function, in accordance with an embodiment of the present invention.

### DETAILED DESCRIPTION OF PREFERRED EMBODIMENTS

Tuning a language model for a particular task can be performed by using prompt tuning, whereby a prompt is added to a given input, causing the language model to provide results that are adapted to the task. A dynamic prompt may be selected, which may vary by position, length, and prompt pool to provide superior results from a pretrained language model.

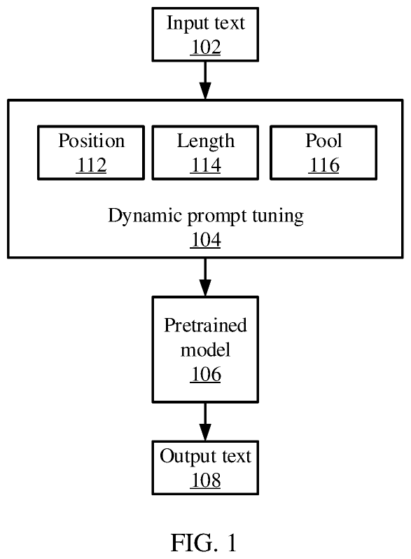

Referring now to FIG. 1, dynamic prompt tuning is shown for a natural language task. An input text 102 is provided, for example by a user's input, in accordance with some natural language task. In some cases the natural language task may include natural language understanding, whereby a model identifies the meaning of the input text 102. In some cases the natural language task may include natural language generation, whereby a model generates a natural language output in accordance with some input. In some cases, a task may include both natural language understanding and natural language generation, for example when a language model is prompted to generate output text having characteristics that are specified by a natural language input. While the present embodiments are described specifically with respect to text prompts, it should be understood that other types of prompts and inputs may be used instead, such as vision prompts with visual features.

Before the input text 102 is applied to a pretrained language model 106, dynamic prompt tuning 104 is performed. The dynamic prompt tuning 104 adds a dynamic prompt to the input text 102, which may vary according to its position 112 relative to the input text 102, its length 114, and/or the pool 116 of prompt options that are being selected from. The modified prompt is used as an input to the pretrained model 106 to generate output text 108. The prompt is selected to cause the output text 108 to be a superior response to the natural language task than would be generated using the input text 102 alone.

For position 112, a prompt may be added as a prefix (prepended to the input text 102) or as a postfix (appended to the input text 102). For a sequence x∈ [symbol] ^{m×d}, the query matrix may be expressed as Q=xW^{Q}∈ [symbol] ^{m×d}, and the key and value matrix are K=xW^{K}∈ [symbol] ^{m×d}V=xW^{V}∈ [symbol] ^{m×d}^{ᵥ} respectively, where m is the number of tokens, d is the token embedding size, and dᵥ is the new embedding size. A prompt P with length l is split into two parts: P=[P₁; P₂], where P₁∈ [symbol] ^{l}^{_{1×d}} and P₂∈ [symbol] ^{l}^{_{2×d}}, where l₁ is the prefix prompt token length and l₂ is the suffix prompt token length. The resulting input becomes x′=[P₁; x; P₂] ∈ [symbol] ^{(l}^{1+m+l}^{₂}^{)×d} and the new key and value matrices become K′=x′W^{K} ∈ [symbol] ^{(l}^{₁}^{+m+l}^{₂}^{)×d} and V′=x′W^{V} ∈ [symbol] ^{(l}^{₁}^{+m+l}^{₂}^{)×d}^{ᵥ} The [;] operator denotes concatenation.

By matrix decomposition:

$${Q}^{′} = [ \begin{matrix}{Q}_{1} \\ Q \\ {Q}_{2}\end{matrix} ]$$

$${K}^{′} = [ \begin{matrix}{K}_{1} \\ K \\ {K}_{2}\end{matrix} ]$$

$${V}^{′} = [ \begin{matrix}{V}_{1} \\ V \\ {V}_{2}\end{matrix} ]$$

where Q₁, K₁ ∈ [symbol] ^{l}^{_{1×d}}, Q₂, K₂ ∈ [symbol] ^{l}^{_{1×d}}, V₁∈ [symbol] ^{l}^{₂}^{×d}^{ᵥ}, and V₂ ∈ [symbol] ^{l}^{₂}^{×d}^{ᵥ}, and Q′ is the concatenation of Q₁, Q, and Q₂.

The sequence x may be expressed as a sequence of n tokens x={x₁, . . . , xₙ}. A pretrained language model 106 may be used to generate an embedding of the tokens X∈ [symbol] ^{n×d} where e is the dimension of the encoded representation. The pretrained model 106 may be any appropriate text processing model, such as a large language model based on transformers. In some cases, a soft prompt P₁ may be prepended to an input X to form the matrix X′=[P; X], where X′ is provided to the model 106 for optimization and where only the parameters of P are optimized while the backbone language model 106 is kept static.

However, using the prefix prompt P₁ may not always be optimal. The prefix intuitively provides extra information for the input sequence and offers an avenue for optimization, but may not be sufficient. Thus the position of the prompt may be selected dynamically, where a position parameter may be learned for different tasks or instances. As above, the prompt may therefore be expressed as P_{prefix}=[P₁, P₂, . . . , P_{dpos}] and P_{postfix}=[P_{dpos+1}, . . . , Pₗ]. Thus, the new input to the language model 106 becomes:

X′=[P_{prefix}; X; P_{postfix}]

where dpos ∈[0, l] is an integer to learned, and where dpos=l corresponds to a prompt which is entirely prefix. Since dpos is categorical, a function POS_{θ} and the Gumbel-Softmax may be used to optimize it. The function POS_{θ} may generate a vector having a length that corresponds to the different positions for the prompt (e.g., 2). The Gumbel-Softmax approach optimizes the output so that it is a binary vector that, if a given dimension of the Gumbel-Softmax's output is a ‘1’, then the value corresponding to that dimension is selected as the position. Given the output of the neural network, α∈ [symbol] ^{l+1}, a binary vector of the same size may be estimated.

For example, to decide dpos, there are (l+1) positions to select from. The values {α₀, . . . , α₁} represent log probabilities {log(p₀), . . . , log(pₗ)} of different insertion positions and a is the output logit of POS_{θ}. Samples can be drawn by drawing samples {g₀, . . . , gₗ} from a Gumbel distribution. In other words, g=− log(− log(z))˜Gumbel, where z is a uniform distribution from 0 to 1. The discrete sample can be produced by adding g to introduce stochasticity:

$$dpos = {arg max}_{i} [ {α}_{i} + {g}_{0} ] , i ∈ \{ 0 , … , l \}$$

$${μ}_{i} = {e}^{\frac{\frac{( {α}_{i} + {g}_{i} )}{τ}}{{\sum }_{\hat{ι} = 0}^{l} {e}^{\frac{( {α}_{\hat{ι}} + {g}_{\hat{ι}} )}{τ}}}} , i ∈ \{ 0 , … , l \}$$

where μᵢ is the calculation of softmax to approximate a discrete selection of the Gumbel distribution.

The argmax operation is non-differentiable, but the softmax can be used as a continuously differentiable approximation to it. The temperature r controls the discreteness. Thus argmax can be used to make the discrete selection on the forward pass, while approximating it with softmax on the backward pass.

An example of the binarization function selects the position with a maximum value of {α₀, α₁, . . . α₁}, but this approach is non-differentiable. The gradients may be propagated through discrete nodes, for example using Gumbel-Softmax sampling. Thus:

$$logit = Gumbel - Softmax ( {POS}_{θ} ( x ) , τ ) \qquad ( x )$$

where t is an annealing temperature adjusted by the total training steps. The logit is an (l+1)-dimensional binary vector where only one element is equal to one and all other elements are zero.

The prompt length may be selected for particular models and tasks, though going beyond 20 tokens tends to give marginal gains. Thus l=20 may be used as a prompt length for most cases. Additional parameters are bought by the small network of POS_{θ} with one liner layer, which may have a size d×(l+1). This instance-dependent position selection may be understood herein as an adaptive position on instance-level selection (adap_ins_pos). In contrast, learning an optimal position for all instances in a task may be performed using a vector v∈ [symbol] ^{l+1} to learn a global best position for all instances within the task, referred to herein as adaptive position on task-level (adap_pos). Then the number of additional parameters may be l+1.

Prompt length 114 provides further options for prompt tuning. Attention masks may be used:

$$Attention = softmax ( \frac{Q * {K}^{T}}{\sqrt{{d}_{k}}} + M ) * v$$

$$where M = \{ \begin{matrix}0 , & mask = 1 \\ - ∞ , & mask = 0\end{matrix}$$

and where Q is the query, K is the key, dₖ is the dimension of the key matrix, and V is a value matrix.

While truncated prompts could be treated as padding tokens with mask=0, the prompt length lᵢ being dynamically updated in the input instance xᵢ means that the logits returned by Gumbel-Softmax cannot be directly applied to the attention mask matrix M, as M cannot provide gradients.

The prompt length 114 may be dynamically learned:

$${l}^{*} = {arg min}_{i} loss ( LM ( [ {\hat{P}}_{i} ; X ] \vert {\hat{P}}_{i} ∈ \{ {\hat{P}}_{1} , … , {\hat{P}}_{l} \} , {\hat{P}}_{i} ∈ {ℝ}^{i \times d} ) )$$

where LM is the language model and loss is a loss function, such as a cross-entropy loss for classification tasks, and where P is the prompt with the selected optimal prompt length. The prompt length l*∈[0, l] is categorical and can be optimized using the Gumbel-Softmax. The length l represents the maximum permissible length for the selection process. The number of additional parameters may be l+1 and d*(l+1) for task- and instance-level, respectively. Models may necessitate fixed input matrix dimensions, so a surrogate strategy may be used. In particular:

$${P}_{new} = [ 0 * {P}_{prefix} ; {P}_{postfix} ]$$

where P_{prefix}∈ [symbol] ^{(l-l}^{_{i)×d}} and P_{postfix}∈ [symbol] ¹^{_{i×d}}. Although the attention score after softmax is not zero, the corresponding value in V is zero.

The prompts themselves may be generated from prompt pools 116. For example, there may be a set of prompt pools Pool={P⁽¹⁾, . . . , p^{(k)}}, where k is the number of pools. Given any input x, a small network Pₒ_{_{θ}} may be learned to get the attention score of every prompt p^{(k)} with respect to x to generate a new prompt:

$${P}_{new} = {\sum }_{i = 1}^{k} {β}_{i} * {P}^{( i )} , β = softmax ( {P}_{{O}_{θ}} ( x ) )$$

In practice, k controls the size of the prompt pool and increased parameters. Since P_{new} depends on a specific input instance, this is referred to herein as an adaptive vector on instance-level approach.

In contrast to hard prompts, which may be predefined text strings, a sequence of vector may be used as a soft prompt. The input text is transformed to embedded vectors before being sent to the first transformer layer. Before that input occurs, prompt vectors are added before or after the input token vectors. The attention score comes from a neural network that takes the input sequence and outputs a weight vector. Rather than having one global prompt vector, there may be K different prompt vectors of a given size. This pool of k different prompt vectors may be randomly initialized, and weights for the vectors may be learned, with the initially random vector values being included in the optimization as well.

The selection of prompt position 112, length 114, and pools 116 can be combined in dynamic prompt tuning 104 to adapt the prompt to the specific input x and task being performed. For example, the dynamic position 112 and prompt pools 116 may be updated together, referred to herein as adaptive instance-vector-position. In another example, dynamic position 112 may first be used to learn the best task-level position so that the instance-level prompt pool 116 may be updated, denoted as adaptive position-instance vector.

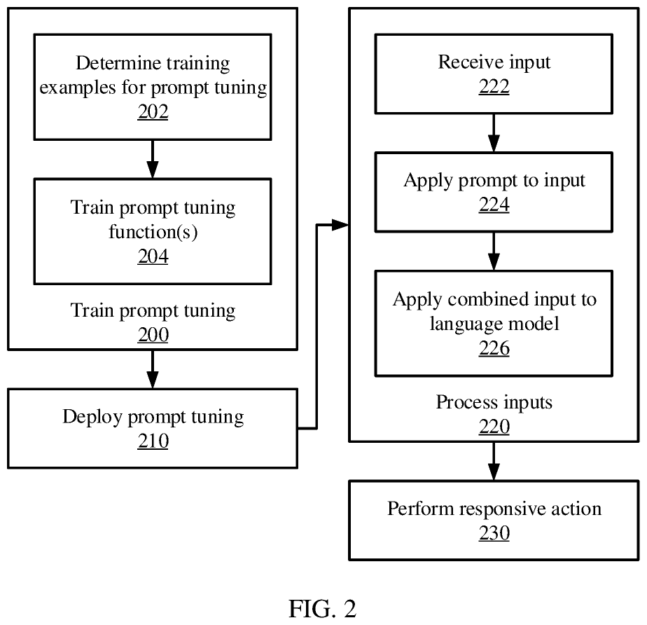

Referring now to FIG. 2, a method for training and using prompt tuning is shown. The overall method includes training prompt tuning 200, deploying prompt tuning 210, processing inputs 220, and performing a responsive action 230. These acts may be performed at different physical locations, using different computing hardware, but in some embodiments one or more of these steps may be performed on a single computing system.

Training the prompt tuning 200 includes determining training examples for prompt tuning 202 and supervised training of the prompt tuning function(s) 204. The training examples may include a set of labeled examples for prompts of differing lengths, positions, and pools, so that the tuning functions can be trained to generate values for these tuning variables that reflect a given task. For example, if the task is sentiment classification, where the input is a sentence and the output is a classification of the sentiment (e.g., a 0 for a negative sentiment or a 1 for a positive sentiment), then the training examples may include pairs of sentences and classifications. The training 204 identifies parameters for the lengths, positions, and pools that cause the language model to provide superior performance for a given language processing task. To train these parameters, a sentence from an example is sent to the model and the output is compared to the associated example classification. A loss function is used to guide a gradient descent for adjusting the prompt parameters to improve the model's performance.

Once the prompt functions have been trained, they may be deployed 210. The deployment 210 may include implementation of a language model for a particular language processing task, along with a prompt tuning system. When processing inputs 220, an input is received 222 in the form of text, for example as a natural language query to the language model. For example, the language model may represent a chatbot in a medical or healthcare context, where a medical professional uses the chatbot to collect information from a patient or to perform a diagnosis. In such cases, the input may be directed to the language model in the form of a question to elicit a particular type of information, or may respond to questions from the chatbot.

Before the input reaches the language model, a tuned prompt is devised and applied to the input 224. This tuning may include any combination of prompt position 112, prompt length 114, and prompt pool 116. Applying the tuned prompt includes adding the text of the tuned prompt to the input at the prescribed prompt position 112 to form a combined input. The combined input is then applied 226 to the language model to generate an output.

Based on the output of the language model, block 230 performs a responsive action. For example, a medical professional may make a decision regarding treatment for a patient based on the response of the language model. In some cases, an input to the language model may prompt the language model to take some automated action, for example when receiving a command or instruction.

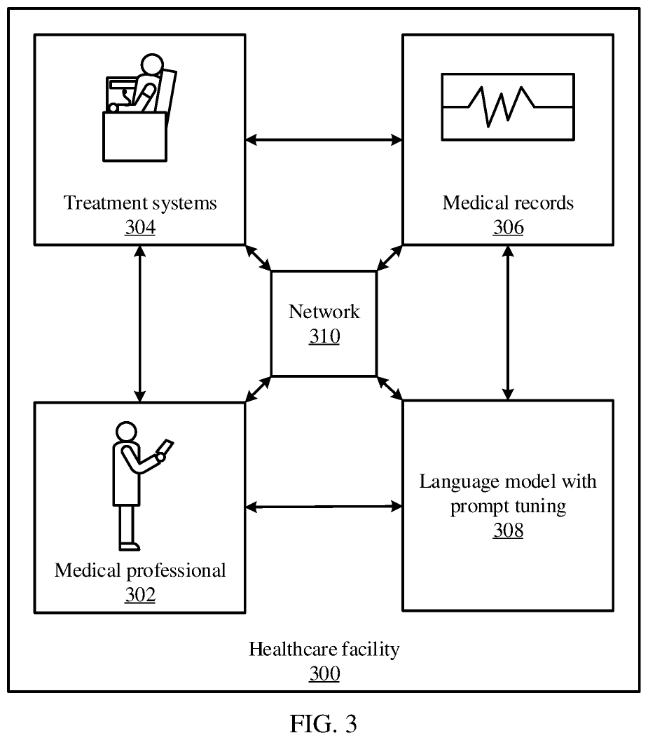

Referring now to FIG. 3, a diagram of review summarization is shown in the context of a healthcare facility 300. A language model with prompt tuning 308 may be used to guide decision-making for medical professionals 302, for example in determining which healthcare products are appropriate to treat a given patient or to use in the healthcare facility 300. The language model with prompt tuning 308 may also or alternatively be used to provide medical consultations for patients, so that the patients can obtain information about their condition and treatment without taking up the time of a medical professional 302.

The healthcare facility may include one or more medical professionals 302 who review information from a patient's medical records 306 to determine their healthcare and treatment needs. The medical records 306 may furthermore be made available to the language model with prompt tuning, so that questions about a patient's particular case can be considered. Treatment systems 304 may furthermore monitor patient status to generate medical records 306 and may be designed to automatically administer and alter treatments as needed. The medical records 306 may be provided as input to the language model to inform the language model's responses to queries. Thus, a patient may ask the language model 308 questions about their particular medical history or current medical state.

Based on information from the medical records 306 and the treatment systems 304, the language model with prompt tuning 308 may conduct a conversation with a patient or medical professional 302 to provide information about the patient. The tuned prompt may serve to adapt a pretrained language model to the medical context without having to fine-tune the model itself. The medical professionals 302 may then make decisions about patient healthcare based on the output of the language model. In some cases, the language model with prompt tuning 308 may automatically provide instructions to the treatment systems, for example in instances where the patient's inputs indicate an urgent need.

The different elements of the healthcare facility 300 may communicate with one another via a network 310, for example using any appropriate wired or wireless communications protocol and medium. Thus the language model with prompt tuning 308 sends responses to patients and medical professionals 302, who may make healthcare decisions in the context of the patient's medical records 306. In some cases, the language model with prompt tuning 308 may be integrated with an automated treatment system 304, which may automatically trigger treatment changes for a patient in response to information gleaned from a conversation. For example, if the patient indicates discomfort or negative side effects from a particular treatment, then the treatment system 304 may automatically cease treatment until a medical professional 302 can review the decision.

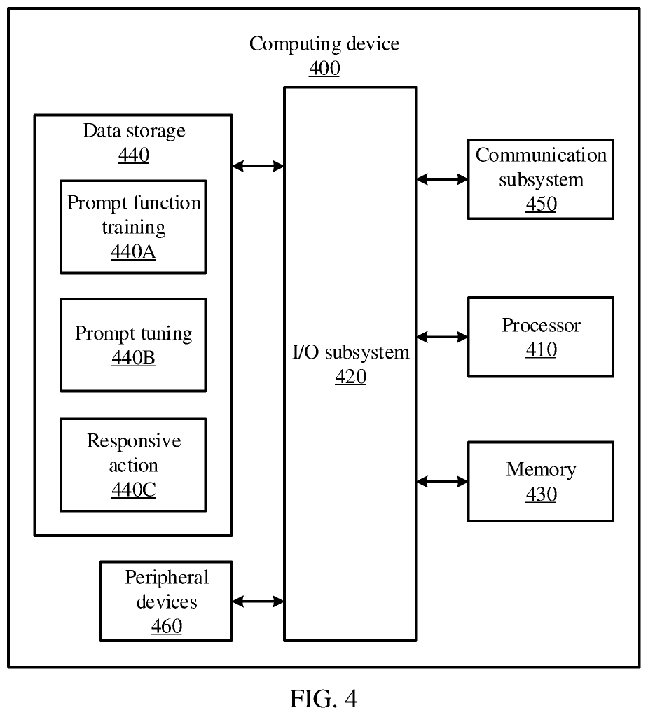

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for training prompt functions, 440B for performing prompt tuning, and/or 440C for performing an automatic action responsive to a language model. Any or all of these program code blocks may be included in a given computing system. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

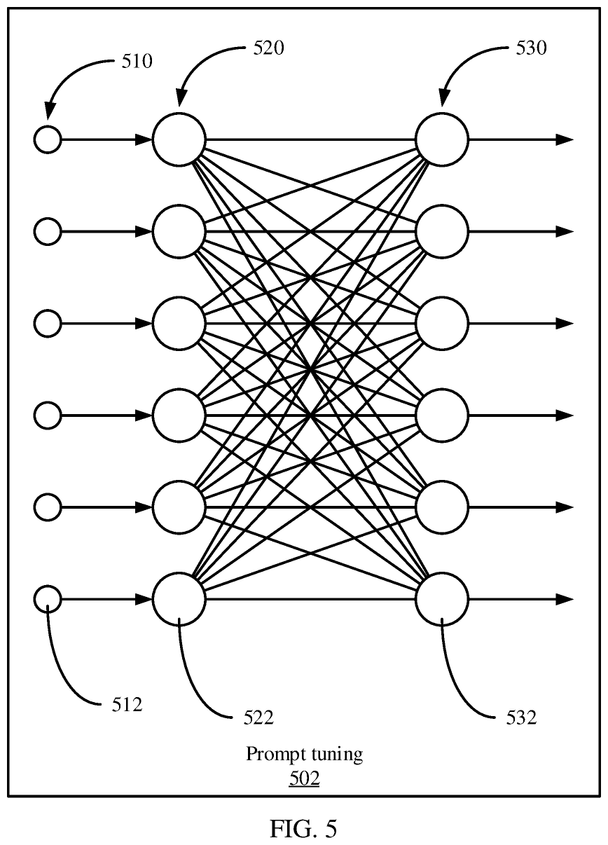

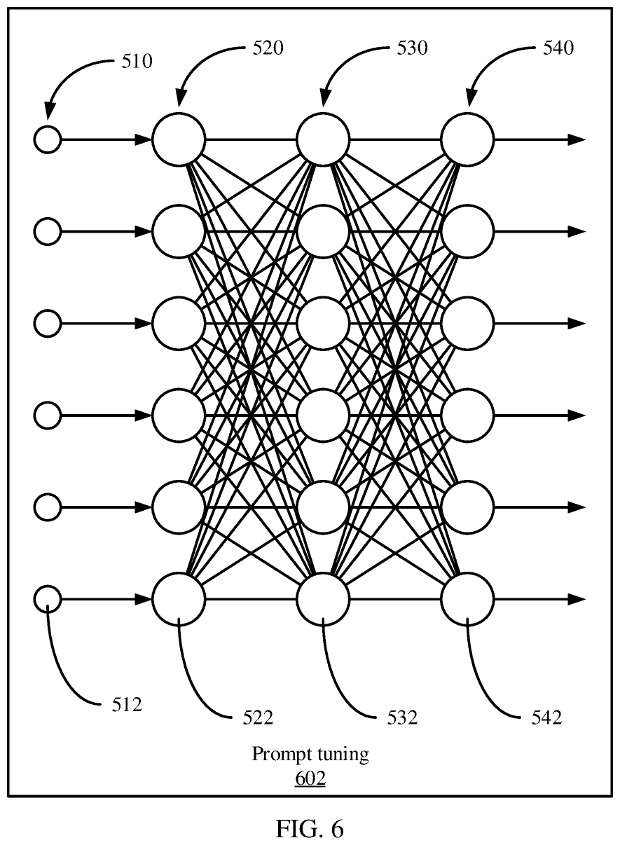

Referring now to FIGS. 5 and 6, exemplary neural network architectures are shown, which may be used to implement parts of the present models, such as the prompt tuning 502 and 602. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 532 in the one or more computation (hidden) layer(s) 530 perform a nonlinear transformation on the input data 512 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/of”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for prompt tuning, comprising:
training a tuning function to set prompt position, prompt length, or prompt pool based on a language processing task;
applying the tuning function to an input query to generate a combined input, with prompt text having the prompt length, being selected according to the prompt pool, and being added to the input query at the prompt position; and
applying the combined input to a language model.

2. The method of claim 1, wherein training the tuning function selects a prompt prefix length and a prompt postfix length, such that the prompt text is includes a prefix part that is prepended to the input query and a postfix part that is appended to the input query.

3. The method of claim 1, wherein training the tuning function selects a length for the prompt text by minimizing a loss function of the language model.

4. The method of claim 1, wherein training the tuning function selects the prompt text according to a weighted sum of prompts in a prompt pool.

5. The method of claim 1, wherein the language model implements a chatbot that has access to patient information for medical decision making in a healthcare setting.

6. The method of claim 5, wherein the input query is from a healthcare professional regarding a patient's condition or treatment, further comprising performing an action responsive to an output of the language model.

7. The method of claim 5, wherein the input query is from a patient regarding a condition or treatment of the patient, further comprising automatically altering the patient's treatment based on an output of the language model.

8. The method of claim 5, wherein the patient information includes medical history information and treatment information.

9. The method of claim 1, wherein the language model is a pretained language model based on transformers.

10. The method of claim 1, wherein training the tuning function performs supervised learning based on a set of training examples for the language processing task.

11. A system for prompt tuning, comprising:
a hardware processor; and
a memory that stores a computer program which, when executed by the hardware processor, causes the hardware processor to:
train a tuning function to set prompt position, prompt length, or prompt pool based on a language processing task;
apply the tuning function to an input query to generate a combined input, with prompt text having the prompt length, being selected according to the prompt pool, and being added to the input query at the prompt position; and
apply the combined input to a language model.

12. The system of claim 11, wherein training the tuning function selects a prompt prefix length and a prompt postfix length, such that the prompt text is includes a prefix part that is prepended to the input query and a postfix part that is appended to the input query.

13. The system of claim 11, wherein the computer program further causes the hardware processor to select a length for the prompt text by minimizing a loss function of the language model.

14. The system of claim 11, wherein the computer program further causes the hardware processor to select the prompt text according to a weighted sum of prompts in a prompt pool.

15. The system of claim 11, wherein the language model implements a chatbot that has access to patient information for medical decision making in a healthcare setting.

16. The system of claim 15, wherein the input query is from a healthcare professional regarding a patient's condition or treatment, further comprising performing an action responsive to an output of the language model.

17. The system of claim 15, wherein the input query is from a patient regarding a condition or treatment of the patient, further comprising automatically altering the patient's treatment based on an output of the language model.

18. The system of claim 15, wherein the patient information includes medical history information and treatment information.

19. The system of claim 11, wherein the language model is a pretrained language model based on transformers.

20. The system of claim 11, wherein the computer program further causes the hardware processor to perform supervised learning of the tuning function based on a set of training examples for the language processing task.
